(19) United States
(12) Patent Application Publication
(10) Pub. No.: US 2025/0319288 A1
(43) Pub. Date: Oct. 16, 2025

(54) ARTICLES AND METHODS FOR BALLOON ANGIOPLASTY

(71) Applicant: Drexel University, Philadelphia, PA (US)

(72) Inventors: John Francis Eberth, Ardmore, PA (US); Tarek Michael Shazly, Columbia, SC (US); Vijaya B. Kolachalama, Weston, MA (US)

(21) Appl. No.: 19/177,170

(22) Filed: Apr. 11, 2025

Related U.S. Application Data
(60) Provisional application No. 63/632,815, filed on Apr. 11, 2024.

(51) Int. Cl. A61M 25/10; A61M 25/00

(52) U.S. Cl. CPC A61M 25/104; A61M 25/10182; A61M 25/10188; A61M 2025/0001; A61M 2025/109; A61M 2205/3344

(57) ABSTRACT

Provided herein is an angioplasty balloon inflation device. The angioplasty balloon inflation device includes a processor; a pressure sensor coupled to the processor; a motor coupled to the processor; and an inflation element including a first end coupled to the motor and a second end configured for coupling to an angioplasty balloon. The motor is configured to adjust the pressure in the angioplasty balloon through the inflation element, the pressure sensor is configured to monitor pressure in the angioplasty balloon, and the processor is configured to control the motor based upon the pressure monitored by the pressure sensor. Also provided herein is a method of performing percutaneous transluminal balloon angioplasty (PTA) using the device.

### CROSS-REFERENCE TO RELATED APPLICATIONS

The present application claims priority under 35 U.S.C. § 119 (c) to U.S. Provisional Patent Application No. 63/632,815, filed Apr. 11, 2024, which application is incorporated herein by reference in its entirety.

### STATEMENT REGARDING FEDERALLY SPONSORED RESEARCH OR DEVELOPMENT

This invention was made with government support under grant number R01-HL159620 awarded by the National Institutes of Health. The government has certain rights in the invention.

### BACKGROUND OF THE INVENTION

Performed nearly a million times per year in the US at $32 k/intervention, catheter-based percutaneous transluminal balloon angioplasty (PTA) is a minimally invasive endovascular procedure used to treat obstructed arteries of the coronary, peripheral, renal, or central vasculature. Despite often integrating stent placement and/or antiproliferative drug delivery (i.e., drug-coated balloons or DCBs), there remains a significant risk of wall injury, restenosis and/or vessel wall dissection/rupture in patients following PTA.

The angioplasty balloon pressurization process is highly nonlinear involving three distinct phases: (1) rapid unfolding and expansion of the balloon, (2) engagement and radial extension of the relatively stiff vessel wall, and (3) rapid expansion and dilation as stenotic plaques yield and the lumen is restored. Using conventional devices, coarse pressure adjustments are made by direct plunger translation while fine-tuning may be performed using a lead screw. In both scenarios, actuation depends on the user's two-handed dexterity and reaction time with little-to-no control over the rate. While it is understandable that high pressures must be developed to reopen the vessel in the presence of stiff plaque, and that these pressures vary on a case-to-case basis, on average, stenosis pressures in the coronary vascular require roughly 4.4±2.3 atm. Despite this, recorded pressures regularly exceed 2-8 times these values. Excessively high pressures and rapid expansion rates, even temporarily, increase the likelihood of vascular cell damage and wall injury leading to dissection and/or restenosis. Frustratingly, these analog step devices are the only designs currently on the market and are used ubiquitously throughout the clinic.

Accordingly, there remains a need in the art for improved articles and methods for balloon angioplasty. The present invention meets this need.

### SUMMARY

In one aspect, an angioplasty balloon inflation device includes a processor; a pressure sensor coupled to the processor; a motor coupled to the processor; and an inflation element including a first end coupled to the motor and a second end configured for coupling to an angioplasty balloon; wherein the motor is configured to adjust the pressure in the angioplasty balloon through the inflation element, the pressure sensor is configured to monitor pressure in the angioplasty balloon, and the processor is configured to control the motor based upon the pressure monitored by the pressure sensor. In some embodiments, adjusting the pressure includes increasing, decreasing, or maintaining a pressurization rate and a magnitude of pressure. In some embodiments, the device is configured and adapted to be operated by one hand.

In some embodiments, the second end is configured for coupling to the angioplasty balloon through a tube. In some embodiments, the pressure sensor is coupled to the tube, between the second end and the angioplasty balloon.

In some embodiments, the device is further configured to record data from operation thereof. In some embodiments, the data includes pressurization rate and magnitude of pressure, as measured by the pressure sensor.

In some embodiments, the processor is further configured to determine when plaque yields while the balloon is inflating within a vessel containing plaque. In some embodiments, the determination is based on at least one of a pressurization magnitude, a pressurization rate, and an infusion volume.

In some embodiments, the device further includes a digital library used in connection with an inflation procedure. In some embodiments, the digital library includes information relating to properties of different angioplasty balloons. In some embodiments, the digital library includes selectable and programmable inflation/deflation/dwell sequences.

In some embodiments, the device is configured to implement an automated inflation procedure. In some embodiments, the processor is configured to automatically control the motor based upon the monitored pressure from the pressure sensor, the automatic control of the motor adjusting the pressure in the angioplasty balloon.

In some embodiments, the device further includes a display coupled to the processor. In some embodiments, the display is configured to show at least one of a measured pressure, a pressure setpoint, a maximum pressure, a dispensed volume, a dwell time, and a pump status indicator.

In some embodiments, the device further includes a storage element configured to record data from operation of the device.

In another aspect, a method of performing percutaneous transluminal balloon angioplasty (PTA) includes providing the device according to any of the embodiments disclosed herein; inserting the angioplasty balloon into a patient; providing a pressurization command to the device, the pressurization command causing the motor to actuate the infusion element and inflate the angioplasty balloon; monitoring a pressure in the angioplasty balloon using the pressure sensor; and automatically adjusting the pressurization command based upon the monitored pressure. In some embodiments, the method further includes automatically adjusting the pressurization command in view of a digital library. In some embodiments, the automatically adjusting step includes maintaining at least one of a pressurization rate and a pressure level within a set range.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIGS. 1A-D show images of a digital balloon inflator. (A) Schematic of a digital balloon inflator. (B) Photographic hardware illustration with angioplasty balloon phantom. (C) Organic Light-Emitting Diode (OLED) pressure and pump control display. (D) Electrical diagram.

FIGS. 2A-C show a schematic and graphs illustrating setup and operation of a digital inflator. (A) Schematic illustrating incremental feedback so the sign difference between the measured and setpoint pressures determines if the syringe pump moves and the direction of travel. (B) Using a balloon angioplasty phantom, the setpoint pressure is easily reached. The dashed box is a zoomed in area at the point the pressure is achieved. (C) Using a balloon angioplasty phantom, the real-time dispensed volume known.

### DETAILED DESCRIPTION OF THE INVENTION

### Definitions

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

The terms “proximal” and “distal” can refer to the position of a portion of a device relative to the remainder of the device or the opposing end as it appears in the drawing. The proximal end can be used to refer to the end manipulated by the user. The distal end can be used to refer to the end of the device that is inserted and advanced and is furthest away from the user. As will be appreciated by those skilled in the art, the use of proximal and distal could change in another context, e.g., the anatomical context in which proximal and distal use the patient as reference, or where the entry point is distal from the user.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION

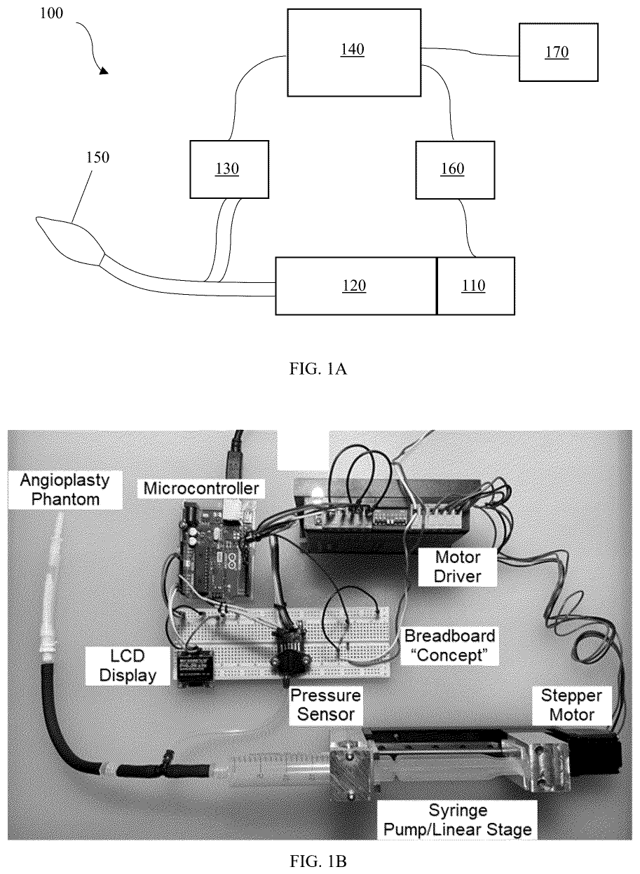

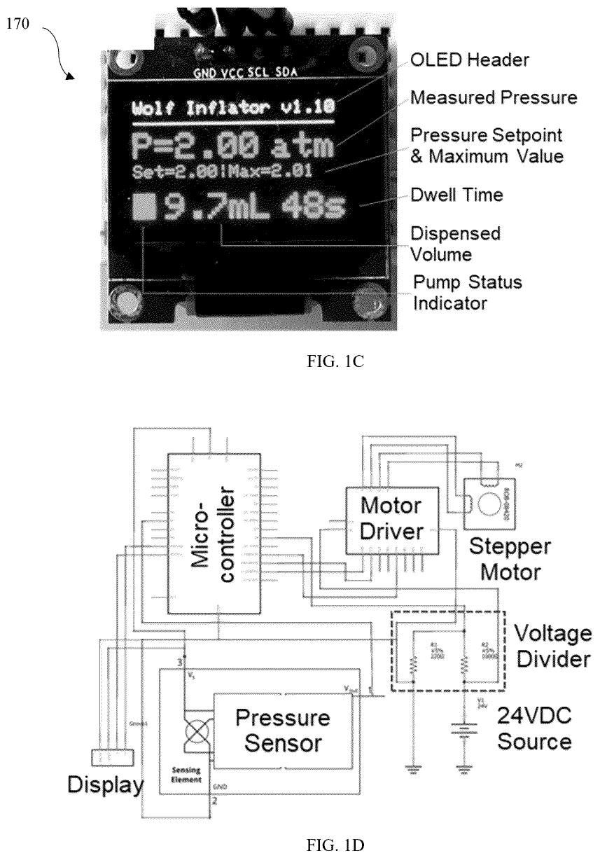

Provided herein are inflation devices for balloon angioplasty. Referring to FIGS. 1A-D, in some embodiments, the device includes a motor 110, an inflation element 120, a pressure sensor 130, and a processor 140. In such embodiments, the motor 110 is configured to drive the inflation element 120, which inflates and/or deflates an angioplasty balloon 150, while the pressure sensor 130 is configured to measure the pressure in the angioplasty balloon 150. The processor 140 receives the pressure measurement from the pressure sensor 130 and adjusts/controls the motor 110 to provide a desired pressure. In some embodiments, the processor 140 is coupled to a motor driver 160, which controls the motor 110 based upon input from the processor 140.

In some embodiments, the device is arranged as a one-handed/handheld device that can be operated with one hand. Accordingly, in some embodiments, the device is portable. As will be appreciated by those skilled in the art, the device can be powered in any suitable manner based upon the device configuration. For example, portable device can include a battery pack and/or pliable power cord, whereas devices with more fixed elements can be coupled directly to a power line and/or outlet.

The inflation element includes any suitable element for providing a desired pressure in the angioplasty balloon. In some embodiments, the inflation element includes any suitable pump or other element having fine tunability. For example, in some embodiments, the inflation element includes a syringe pump, an electronically actuated screw pump, a piston pump, a diaphragm pump, a peristaltic pump, a gear pump, or any other suitable element for inflating the angioplasty balloon. The inflation element can be coupled to the angioplasty balloon in any suitable manner, such as, but not limited to, through tubing (e.g., catheter).

As will be appreciated by those skilled in the art, the motor can be selected based upon the specific inflation element being used. For example, in some embodiments, the motor includes a stepper motor with a driving lead screw configured to actuate the syringe pump (i.e., increase or decrease the volume in the syringe pump). Other suitable motors include, but are not limited to, a magnetic linear motor, a DC motor with positional feedback, or any other suitable electronically actuated motor. In some embodiments, the motor is coupled to the inflation element (e.g., mechanically, electrically). Alternatively, in some embodiments, the motor and the inflation element are formed as a single unit.

In some embodiments, the motor is coupled to any suitable motor driver for regulating speed and/or direction of the motor. For example, in some embodiments, the motor driver includes a 2-phase stepper motor driver using pulse-width-modulation (PWM) to regulate speed, control the direction, and/or enable/disable functionality of the motor. In some embodiments, the inflation element, the motor, and/or the motor driver are configured to provide pressures of up to 5 ATM, up to 10 ATM, up to 15 ATM, up to 20 ATM, up to 25 ATM, or any combination, sub-combination, range, or sub-range thereof.

The pressure sensor includes any suitable sensor for measuring pressure in an angioplasty balloon coupled to the inflation element. Suitable pressure sensors can include any suitable format for outputting an electronic signal (e.g., voltage, current, frequency, etc.) that can be measured by the processor. In some embodiments, the pressure sensor includes any sensor capable of sensing pressures at a level at least as high as the maximum pressure achieved by the device. For example, in some embodiments, the pressure sensor includes a 3/5 VDC, 700kPA pressure sensor (e.g., MPX5700AP from NXP®). Additionally or alternatively, in some embodiments, the pressure sensor includes multiple pressure sensors configured to provide improved resolution across a full span. In some embodiments, the pressure sensor includes an integrated amplifier, such as, but not limited to, a dual, 36-V, 1.2-MHz, 3-mV offset voltage operational amplifier (e.g., LMN358 from Texas Instruments®). As will be appreciated by those skilled in the art, the pressure sensor can be coupled to the inflation element, tubing, and/or angioplasty balloon in any suitable manner for measuring the pressure. For example, in some embodiments, the pressure sensor is coupled to the tubing between the inflation element and the angioplasty balloon.

The processor includes any suitable processor for receiving pressure measurements from the pressure sensor and controlling/adjusting the motor based upon the measured pressure. In some embodiments, the processor includes a microcontroller. For example, one suitable processor includes, but is not limited to, an Arduino® Uno® microcontroller. Although described herein primarily with respect to an Arduino® microcontroller, as will be appreciated by those skilled in the art, the disclosure is not so limited and expressly includes any other suitable microcontroller or processor. The processor can be coupled to the pressure sensor and motor/motor driver through any suitable manner such as, but not limited to, directly, through a printed computer board (PCB), a bread board, or any other suitable manner.

In some embodiments, the device 100 also includes a display 170. The display is coupled to the processor in any suitable manner as described elsewhere herein and includes any suitable display for showing status/properties of the device. Suitable displays include, but are not limited to, a liquid crystal display (LCD), light emitting diode (LED), organic LED (OLED), touchscreen, any other suitable display type, and/or combinations thereof. In some embodiments, the display is configured to show one or more of a header, measured pressure, pressure setpoint, maximum pressure, dispensed volume, dwell time, and/or pump status indicator. In some embodiments, the pump status indicator shows a symbol indicating whether the pump is infusing or increasing pressure (e.g., ↑), withdrawing or decreasing pressure (e.g., ↓), or maintaining/stopped (e.g., ▪).

Additionally or alternatively, in some embodiments, the device includes a data storage element. Suitable data storage elements include, integrated memory, hard drives, removable memory, remote storage (e.g., remote servers, cloud storage), or any other suitable element for receiving and storing data. In some such embodiments, the device records and stores data during each procedure, such as, but not limited to, inflation rate, maximum pressure, vessel reopening, and/or any other suitable property relating to the operation of the device and/or the procedure. In some embodiments, this data can be used to evaluate and/or standardize procedures and/or improve clinical outcomes.

In some embodiments, the device is configured to operate through an “incremental control” control strategy. In such embodiments, after each iteration of the feedback loop, the motor moves one predefined incremental step in either direction or not at all when within a predefined threshold. For example, in some embodiments, the device is configured to automatically provide precise balloon inflation up to a set point, without overshooting a maximum pressure, by measuring the pressure and incrementally adjusting the motor in real-time. The pressure setpoint and/or maximum pressure can be determined/input to the device between each procedure, or a standard/default set of values can be adjustably pre-set. For example, in some embodiments, a user inputs a target pressure and/or maximum (not to exceed) pressure and the device controls inflation of the angioplasty balloon based upon the user input. Additionally or alternatively, in some embodiments, the device automatically determines a target pressure and/or maximum pressure based upon data stored on the device (e.g., a lookup table using data taken from the manufacturer).

The device can also include one or more security features. For example, in some embodiments, the device includes alarm/warning indicators and/or hardware/software fail safes. The alarm/warning indicators can include any alarm/warning related to the procedure. Such alarms/warnings include, but are not limited to, inflation rate alarms (e.g., inflation rate is higher or lower than expected), inflation amount alarms (e.g., pressure is too high or low for volume infused), plaque yield alarms (e.g., approaching maximum pressure without desired inflation), or any other suitable alarm/warning. The alarms/warnings can be auditory, visual, or both. For example, in some embodiments, the alarms/warnings are part of the onboard display and/or linked to Bluetooth. Suitable hardware/software fail safes include, but are not limited to, limits on inflation rate and/or amount. For example, in some embodiments, the hardware/software fail safes includes mechanical or digital pressure release valves, automatic electronic shutoffs, or any other suitable fail safe.

Also provided herein are methods of performing percutaneous transluminal balloon angioplasty (PTA). In some embodiments, the method includes inputting a pressure setpoint to the device according to any of the embodiments disclosed herein, inserting the angioplasty balloon into the vessel to be reopened, activating the device to inflate the balloon, reopening the vessel, deflating the balloon, and withdrawing the balloon from the vessel. In such embodiments, the device automatically monitors and controls the inflation to provide precise control over the process (e.g., rate, amount, and/or maintenance of balloon inflation). For example, in some embodiments, the device controls the inflation pressure to within ±0.01 ATM. As will be appreciated by those skilled in the art, the specific pressure range can be adjusted depending upon the sensitivity of the pressure sensor itself. In some embodiments, the pressure setpoint is preset, such that inputting the pressure setpoint is not separately required. Additionally or alternatively, in some embodiments, the device includes selectable and programmable inflation/deflation/dwell sequences.

In some embodiments, the device is configured to detect plaque yield during the PTA procedure. For example, in some embodiments, the device is configured to calculate diameter of the balloon based upon infused volume. In some such embodiments, the device also includes and/or provides access to a balloon-catheter library. The balloon-catheter library includes information regarding individual angioplasty balloon properties, such that the device can automatically calculate balloon diameter for different balloon types. In some embodiments, the device automatically calculates the balloon diameter with a ±2% error. Accordingly, the device disclosed herein is compatible with diverse balloon-catheter types and manufacturers.

Additionally or alternatively, in some embodiments, the device is configured to provide enhanced drug transfer. For example, in some embodiments, the angioplasty balloon includes a coating configured to release one or more therapeutics therefrom. In such embodiments, the device can be used to administer drug-coated balloon (DCB) therapy, where a drug (e.g., paclitaxel (PTX)) is delivered through inflation of the angioplasty balloon. This delivery can be through a linear and/or non-linear interface model. Various aspects of DCB therapy and linear/non-linear interface models are discussed in Shazly T, Torres WM, Secemsky EA, Chitalia VC, Jaffer FA, Kolachalama VB. Understudied factors in drug-coated balloon design and evaluation: A biophysical perspective. Bioeng Transl Med. 2023; 8 (1): c10370. doi: 10.1002/btm2.10370; Shazly, T., Uline, M., Webb, C., Pederson, B., Eberth, J. F., and Kolachalama, V. B. (Sep. 4, 2023). Novel Payloads to Mitigate Maladaptive Inward Arterial Remodeling in Drug-Coated Balloon Therapy. ASME. J Biomech Eng. December 2023; 145 (12): 121004. doi.org/10.1115/1.4063122; and Shazly T, Eberth JF, Kostelnik CJ, Uline MJ, Chitalia VC, Spinale FG, Alshareef A, and Kolachalama VB. Hydrophilic Coating Microstructure Mediates Acute Drug Transfer in Drug-Coated Balloon Therapy. ACS Appl. Bio Mater. 2024, 7, 5, 3041-3049, which are incorporated herein by reference in their entirety. During DCB, the efficacy of drug deposition is highly dependent on the contact pressure between the balloon and the surface being contacted. By expanding the DCB in a consistent/controlled manner through precisely controlled and monitored infusion, the device described herein provides even, consistent, and efficient drug delivery/transfer during DCB therapy. The processor can also be coupled to linear and/or non-linear interface models, further enhancing drug transfer from the DCB.

By precisely controlling, recording, and maintaining pressurization rate and magnitude, the devices and methods disclosed herein reduce vessel damage risk, enhance drug transfer, monitor procedures, and minimize reintervention rates in PTA therapy. Additionally, as compared to existing devices, the devices and methods disclosed herein are more precise; can control rate as well as magnitude; are sensitive to plaque yielding, thus limiting overshoot; can record pressure and volume data in real time; are programmable so that the pressurization profiles can be tuned; can be operated with one-hand; are digital; minimize operator error and subjectivity; and/or enhance even drug distribution from balloons. Furthermore, the devices and methods disclosed herein provide critical intra-procedural data to the end user (cardiologist), hospitals, and insurers, as well as value-based care organizations.

Although the invention has been described in terms of exemplary embodiments, it is not limited thereto. Accordingly, the appended claims should be construed broadly to include other variants and embodiments of the invention which may be made by those skilled in the art without departing from the scope and range of equivalents of the invention. This disclosure is intended to cover any adaptations or variations of the embodiments discussed herein.

The following examples further illustrate aspects of the present invention. However, they are in no way a limitation of the teachings or disclosure of the present invention as set forth herein.

### EXAMPLES

### Example 1

### Unmet Clinical Need

Catheter-based percutaneous transluminal balloon angioplasty (PTA) is a minimally invasive endovascular procedure used to treat obstructed arteries of the coronary, peripheral, renal, or central vasculature. Despite often integrating stent placement and/or antiproliferative drug delivery (i.e., drug-coated balloons (DCBs)), there remains a significant risk of restenosis and/or vessel wall dissection/rupture in patients following PTA. Failed PTAs can be life-threatening and reintervention is costly. While there are numerous reasons for adverse PTA outcomes (e.g., genetic disorders, diabetes, essential hypertension), it is submitted herein that higher-fidelity control of the balloon pressurization and vessel dilation process will minimize the risk of intra-procedural vessel damage and post-procedural adverse vessel remodeling, and thus enhance both short- and long-term outcomes. Additionally, the digital capture and storage of inflation/deflation data enable procedural standardization, improve clinical protocols, elucidate determinants of interventional success and failure, and direct insurer policies.

During PTA, an uninflated (sheathed) balloon is attached to a catheter and gently guided into place within the stenotic lesion of an occluded vessel. Once positioned, the sheath is removed and fluid is injected through a catheter and into the balloon using an inflation device, typically in the form of a manually actuated, high-pressure syringe pump. The net result is an increase in balloon diameter as the targeted lesion is compressed and the vessel lumen restored. After the transient (up to 2 min) inflation period, the balloon is deflated and the catheter is carefully extracted from the patient. The mechanical processes underlying current balloon deployment are highly discontinuous with three distinct phases: (1) rapid balloon unfolding and expansion, (2) engagement and slow extension of the comparatively stiff vessel wall, and (3) rapid expansion and dilation as stenotic plaques yield and the lumen is restored.

Further balloon expansion is limited in part by the tissue, but mostly by the balloon's engineered material characteristics at a point designated as the “nominal” diameter. These nominal values are provided in the manufacturer's datasheets and generally selected to match the lumen immediately downstream of the stenosis. So-called non-compliant balloons (NCBs) are meant to limit expansion-related damage, yet this designation is somewhat misleading since all balloons deform predictably beyond this point. Semi-compliant (SCB) and fully-compliant (FCB) balloons, on the other hand, are sometimes used as an assessment tool to probe the lesion's properties, or used for dilation when the vessel or lesion shape is non-uniform, or when repeated dilations are necessary, such as in preparation for stent implantation. These routinely undergo 8-20% dilation beyond the nominal value. Thus, high-fidelity inflation control in all balloon types would be advantageous.

While it is understandable that high pressures may be required to reopen vessels in the presence of heterogeneously stiff plaques, and these pressures vary on a case-to-case basis, on average, resolving stenosis in the coronary vasculature require roughly a 4.4+2.3 atm balloon inflation pressure. Despite this, recorded operating pressures in clinical deployments regularly exceed 8 times these values. Excessively high pressures causing overextension and too rapid expansion rates, even temporarily, accentuate vascular cell damage and wall injury. While under-sizing/-expansion will not mitigate restenosis, over-sizing/-expansion promotes dissection, thrombosis, necrosis, inflammation, and restenosis. Likewise, the rate of expansion affects procedural outcomes with slower, controlled dilations, preferred over rapid ones.

Using conventional devices, coarse pressure adjustments are made by direct plunger translation while fine-tuning may be performed using a lead screw. In both scenarios, actuation depends on the user's two-handed dexterity and reaction time with little-to-no control over the rate. As such, conventional balloon angioplasty devices have limited fidelity to decipher optimal versus sub-optimal expansion, and there is no intra-procedural aid to the clinician in this deterministic aspect of PTA. Moreover, the balloon type and/or material description in retrospective, and large-cohort studies are frequently omitted. Therefore, key clinical data (summarized in Table 1) on pressurization alone is sparse and context-specific. Despite the ubiquity of PTA, the mechanism of action and tissue-device interactions during the inflation process are surprisingly poorly understood, hindering device modernization and even emphasizing efficiency (speed of inflation) over effectiveness (procedural outcome). Such misalignment can partially explain procedure-related untoward outcomes such as non-uniform vessel reopening, under- or over-expansion, and non-uniform drug delivery in DCB applications. There is a clear disconnect between product marketing, procedural standardization, and clinical outcomes that can be enhanced by modernizing the process.

TABLE 1
Surgical outcomes due to controllable angioplasty
balloon inflation parameters

| Inflation | Outcome | |
|---|---|---|
| Parameter | (incidence) | Model |
| Over-size/expansion | Dissection (37%) | Human Cor. A (n = 120) [14] |
| | Restenosis (19%) | |
| | Dissection (7.1%) | Human Cor. A (n = 336**) [15] |
| | Other* (7.7%) | |
| | Dissection (92%) | Rabbit Fem. A (n = 35) [16] |
| | Thromb. (69%) | |
| | Inflamm. (66%) | |
| Rapid Inflation | Dissections (59%) | Human Cor. A (n = 72) [17] |
| | Other* (19%) | |
| | Restenosis (36%) | Human Cor. A (n = 234) [9] |
| | Other* (37%) | |
| | Dissection (4.8%) | Human Cor. A (n = 1248) [18] |
| | Other* (9%) | |

*Includes: acute closure, urgent CABG, early failure, side-branch loss, myocardial infarction
**Study halted due to safety concerns

### Device Development

A biophysical contact model was developed that computes interfacial mechanical interactions during DCB deployment and showed that inflation pressure modulates drug transfer to the vessel wall for a range of candidate devices. Thus, for both the inherent risk of vessel damage across PTA applications and the drug delivery kinetics in DCBs, enhanced control of balloon inflation has been identified as an unmet clinical need that is address by the articles and methods disclosed herein. Moreover, digital reporting of both pressure and displaced volume enables correlation of overextension with both acute angiographic (e.g., dissection, lesion compression) and long-term (e.g., restenosis, MI) clinical outcomes.

Accordingly, to improve outcomes following percutaneous transluminal balloon angioplasty (PTA) and provide a means for data collection, described herein is a novel, catheter-based, electro-mechanical digital inflation and monitoring device to replace conventional manually-actuated analog devices. The device described herein displaces current inflators by uniquely providing quantified volume dispensing, digitally-controlled precision pressurization, and through enabling procedural standardization to better patient outcomes. Importantly, this technology provides critical intra-procedural data to the end user (cardiologist), hospitals, and insurers, as well as value-based care organizations, thereby providing an entirely new dimension to standardize, evaluate, and continuously improve the interventional procedure and corresponding clinical outcomes.

The device described herein includes: (a) digital LED display, (b) pressure, volume, & event data storage, (c) plaque yield detection, (d) feedback microprocessor control of inflation pressure (±0.01 ATM) and inflation rate, (e) selectable and programmable inflation/deflation/dwell sequences, (f) alarm & warning indicators, (g) single-handed, ergonomic, intuitive controls, and (h) hardware/software fail safes. Additionally, the device is (i) compatible with diverse balloon-catheter types & manufacturers, (j) configured to calculate diameter from infused volume and embedded balloon-catheter library (+2% error), (k) safe to 25 atm, and/or (1) portable & powered,

A prototype of the hardware, display, and electrical wiring is shown in FIGS. 1B-D. This digital inflator prototype includes the electromechanical components of the device and was tested using an angioplasty balloon phantom. A 3/5 VDC, 700 kPA integrated pressure sensor (MPX5700AP) with an LMN358 amplifier provides feedback information to a programmable Arduino Uno microcontroller in terms of the difference between the measured and setpoint pressures. Custom-written software within the Arduino Integrated Development Environment (IDE) is used to process, record, and output signals to the motor driver.

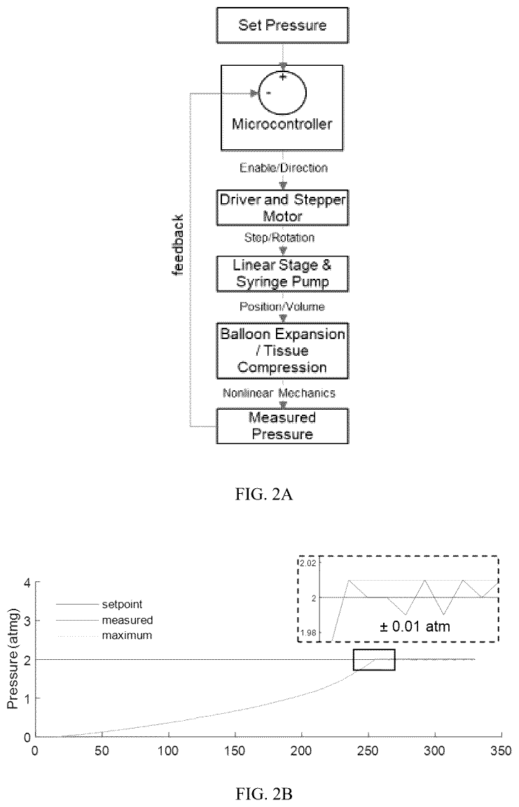

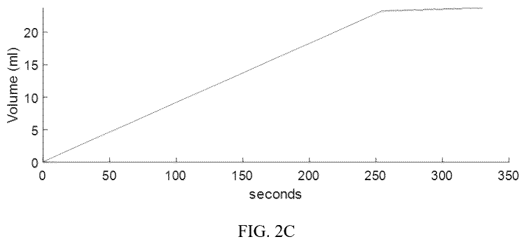

The control strategy currently employed is called “incremental control” because, after each iteration of the feedback loop, the motor moves one predefined incremental step in either direction or not at all when within a predefined threshold. A general interpretation of the control strategy is illustrated diagrammatically in FIG. 2A. The microcontroller interfaces with a 2-phase stepper motor driver using pulse-width-modulation (PWM) to regulate speed and likewise contains logic inputs to control the direction and enable/disable functionality. A 24 VDC, 1.8-degree NEMA11 stepper motor and precision 100 mm linear stage with T6x1 driving lead screw is used to increase or decrease the volume in a syringe pump connected to the catheter balloon and tubing. The increase in volume within the balloon-catheter system results in measurable equilibrium pressures as compliance in the system gradually decreases. A regulated 24 VDC power supply (not shown) energizes the motor driver. A simple voltage divider circuit is made out of 220 and 1000 Ohm resistors to create <5 VDC for logic-level input to the microcontroller when the pump is receiving power.

The syringe pump currently includes any 5-50 ml syringe and custom machined aluminum fittings and mounting hardware. An angioplasty phantom, consisting of a rubber glove inside a compliant tube, is not part of the invention but is used to simulate the nonlinear interaction between the balloon and the vessel wall. An Organic Light-Emitting Diode (OLED) display locally reports the measured gauge pressure in atmospheres, the setpoint pressure (Set)±0.01, and the maximum (Max) pressure recorded during the procedure. A pump status indicator on the display further indicates whether the pump is actively infusing ↑, withdrawing ↓, or maintaining ▪, based on deviation from the setpoint with the latter condition shown here. The dispensed volume and dwell time at the desired pressure is displayed. All elements connect to a breadboard for prototyping purposes only. The rate, precision, and control over overshoot and sensitivity to tissue plaque yielding can be enhanced by using different control strategies.

The device described herein is capable of precision infusion and balloon pressurization up to 4 atm through digitization and feedback control using the electro-mechanical hardware. Because the pressure source uses positive displacement and the feedback control information is sent to a high-speed stepper motor, overinflation is avoided. For example, using a balloon angioplasty phantom, a 2 atm (gauge) setpoint pressure was easily reached in 250 seconds from 0 atm starting point with only a 0.01 atm overshoot (FIG. 2B). The steady pressure was within 0.01 atm of the target. The real-time inflation volume is also shown for this test and can be used to estimate dilation (FIG. 2C). Although not shown, a maximum of 4 atm was reached using off-the-shelf fittings and syringe materials. Moreover, data is stored and pressure profiles are tunable and repeatable thus enabling quantitative studies of device efficacy. This technology will be amenable to a wide spectrum of angioplasty balloons with various operating features (i.e., balloon sizes, compliances).

### EQUIVALENTS

Although preferred embodiments of the invention have been described using specific terms, such description is for illustrative purposes only, and it is to be understood that changes and variations may be made without departing from the spirit or scope of the following claims.

What is claimed is:

1. An angioplasty balloon inflation device comprising:
a processor;
a pressure sensor coupled to the processor;
a motor coupled to the processor; and
an inflation element comprising:
a first end coupled to the motor; and
a second end configured for coupling to an angioplasty balloon;
wherein the motor is configured to adjust the pressure in the angioplasty balloon through the inflation element;
wherein the pressure sensor is configured to monitor pressure in the angioplasty balloon; and
wherein the processor is configured to control the motor based upon the pressure monitored by the pressure sensor.

2. The device of claim 1, wherein the second end is configured for coupling to the angioplasty balloon through a tube.

3. The device of claim 2, wherein the pressure sensor is coupled to the tube, between the second end and the angioplasty balloon.

4. The device of claim 1, wherein the device is further configured to record data from operation thereof.

5. The device of claim 4, wherein the data includes pressurization rate and magnitude of pressure, as measured by the pressure sensor.

6. The device of claim 1, wherein adjusting the pressure includes increasing, decreasing, or maintaining a pressurization rate and a magnitude of pressure.

7. The device of claim 1, wherein the device is configured and adapted to be operated by one hand.

8. The device of claim 1, wherein the processor is further configured to determine when plaque yields while the balloon is inflating within a vessel containing plaque.

9. The device of claim 8, wherein the determination is based on at least one of a pressurization magnitude, a pressurization rate, and an infusion volume.

10. The device of claim 1, further comprising a digital library used in connection with an inflation procedure.

11. The device of claim 10, wherein the digital library includes information relating to properties of different angioplasty balloons.

12. The device of claim 10, wherein the digital library includes selectable and programmable inflation/deflation/dwell sequences.

13. The device of claim 1, wherein the device is configured to implement an automated inflation procedure.

14. The device of claim 13, wherein the processor is configured to automatically control the motor based upon the monitored pressure from the pressure sensor, the automatic control of the motor adjusting the pressure in the angioplasty balloon.

15. The device of claim 1, further comprising a display coupled to the processor.

16. The device of claim 15, wherein the display is configured to show at least one of a measured pressure, a pressure setpoint, a maximum pressure, a dispensed volume, a dwell time, and a pump status indicator.

17. The device of claim 1, further comprising a storage element configured to record data from operation of the device.

18. A method of performing percutaneous transluminal balloon angioplasty (PTA), the method comprising:
providing the device according to claim 1;
inserting the angioplasty balloon into a patient;
providing a pressurization command to the device, the pressurization command causing the motor to actuate the infusion element and inflate the angioplasty balloon;
monitoring a pressure in the angioplasty balloon using the pressure sensor; and
automatically adjusting the pressurization command based upon the monitored pressure.

19. The method of claim 18, further comprising automatically adjusting the pressurization command in view of a digital library.

20. The method of claim 18, wherein the automatically adjusting step includes maintaining at least one of a pressurization rate and a pressure level within a set range.
